SEQUENCE LISTING (17 sequences)

SEQ ID NO: 1 - protein, 495 aa, synthetic construct
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQSHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 2 - protein, 495 aa, synthetic construct
MTSRDGYQWTPETGLTQGVPSLGVISPPTNIEDTDKDGPWDVIVIGGGYCGLTATRDLTVAGLKTLLLEARDRIGGRSWSSNIDGYPYEMGGTWVHWHQSHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 3 - protein, 495 aa, synthetic construct
MTSRDGYQWTPETGLTQGVPSLGVISPPTNIEDTDKDGPWDVIVIGGGYCGLTATRDLTVAGFKVLLLEARDRIGGRSWSSNIDGYPYEMGGTWVHWHQSHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 4 - protein, 495 aa, synthetic construct
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQPHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 5 - protein, 495 aa, synthetic construct
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQSHVWREITRYKMHNALSPSFNFSRGVNHFQLRTNPTTSTYMSHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 6 - protein, 495 aa, synthetic construct
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQSHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMCGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 7 - protein, 495 aa, synthetic construct
MTSRDGYQWTPETGLTQGVPSLGVISPPTNIEDTDKDGPWDVIVIGGGYCGLTATRDLTVAGLKVLLLEARDRIGGRSWSSNIDGYPYEMGGTWVHWHQSHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 8 - protein, 495 aa, synthetic construct
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQPHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMCGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 9 - protein, 495 aa, synthetic construct
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQSHVWREITRYKMHNALSPSFNFSRGVNHFQLRTNPTTSTYMSHEAEDELLRSALHKFTNVDGTNGRTVLPFPHDMFYVPEFRKYDEMSYSERIDQIRDELSLNERSSLEAFILLCSGGTLENSSFGEFLHWWAMCGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 10 - protein, 495 aa, synthetic construct
MTSRDGYQWTPETGLTQGVPSLGVISPPTNIEDTDKDGPWDVIVIGGGYCGLTATRDLTVAGLKVLLLEARDRIGGRSWSSNIDGYPYEMGGTWVHWHQPHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 11 - protein, 495 aa, synthetic construct
MTSRDGYQWTPETGLTQGVPSLGVISPPTNIEDTDKDGPWDVIVIGGGYCGLTATRDLTVAGLKVLLLEARDRIGGRSWSSNIDGYPYEMGGTWVHWHQSHVWREITRYKMHNALSPSFNFSRGVNHFQLRTNPTTSTYMSHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 12 - protein, 495 aa, synthetic construct
MTSRDGYQWTPETGLTQGVPSLGVISPPTNIEDTDKDGPWDVIVIGGGYCGLTATRDLTVAGLKVLLLEARDRIGGRSWSSNIDGYPYEMGGTWVHWHQSHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMCGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 13 - protein, 495 aa, synthetic construct
MTSRDGYQWTPETGLTQGVPSLGVISPPTNIEDTDKDGPWDVIVIGGGYCGLTATRDLTVAGFKTLLLEARDRIGGRSWSSNIDGYPYEMGGTWVHWHQPHVWREITRYKMHNALSPSFNFSRGVNHFQLRTNPTTSTYMSHEAEDELLRSALHKFTNVDGTNGRTVLPFPHDMFYVPEFRKYDEMSYSERIDQIRDELSLNERSSLEAFILLCSGGTLENSSFGEFLHWWAMCGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 14 - protein, 495 aa, synthetic construct
MTSRDGYQWTPETGLTQGVPSLGVISPPTNIEDTDKDGPWDVIVIGGGYCGLTATRDLTVAGLKVLLLEARDRIGGRSWSSNIDGYPYEMGGTWVHWHQPHVWREITRYKMHNALSPSFNFSRGVNHFQLRTNPTTSTYMSHEAEDELLRSALHKFTNVDGTNGRTVLPFPHDMFYVPEFRKYDEMSYSERIDQIRDELSLNERSSLEAFILLCSGGTLENSSFGEFLHWWAMSGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 15 - protein, 495 aa, synthetic construct
MTSRDGYQWTPETGLTQGVPSLGVISPPTNIEDTDKDGPWDVIVIGGGYCGLTATRDLTVAGLKVLLLEARDRIGGRSWSSNIDGYPYEMGGTWVHWHQPHVWREITRYKMHNALSPSFNFSRGVNHFQLRTNPTTSTYMTHEAEDELLRSALHKFTNVDGTNGRTVLPFPHDMFYVPEFRKYDEMSYSERIDQIRDELSLNERSSLEAFILLCSGGTLENSSFGEFLHWWAMCGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 16 - protein, 495 aa, synthetic construct
MTSRDGYQWTPETGLTQGVPSLGVISPPTNIEDTDKDGPWDVIVIGGGYCGLTATRDLTVAGLKVLLLEARDRIGGRSWSSNIDGYPYEMGGTWVHWHQSHVWREITRYKMHNALSPSFNFSRGVNHFQLRTNPTTSTYMSHEAEDELLRSALHKFTNVDGTNGRTVLPFPHDMFYVPEFRKYDEMSYSERIDQIRDELSLNERSSLEAFILLCSGGTLENSSFGEFLHWWAMCGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

SEQ ID NO: 17 - protein, 495 aa, synthetic construct
MTSRDGYQWTPETGLTQGVPSLGVISPPTNIEDTDKDGPWDVIVIGGGYCGLTATRDLTVAGLKVLLLEARDRIGGRSWSSNIDGYPYEMGGTWVHWHQPHVWREITRYKMHNALSPSFNFSRGVNHFQLRTNPTTSTYMSHEAEDELLRSALHKFTNVDGTNGRTVLPFPHDMFYVPEFRKYDEMSYSERIDQIRDELSLNERSSLEAFILLCSGGTLENSSFGEFLHWWAMCGYTYQGCMDCLISYKFKDGQSAFARRFWEEAAGTGRLGYVFGCPVRSVVNERDAVRVTARDGREFAAKRLVCTIPLNVLSSVHFSPPLSPQRMAAANIGHVNQCVKVHAEVSCPDMRSWSGISYPFNKLAYAIGDGTTPAGNTHIVCLGGAHNHIQPEEDVEATKMAVENMSPGNMDIKRLVFHNWCKDEFAKGAWFFAPPQLLSKSLDELRCRHGNVLFANSDWALGWRGFIDGAIEEGTRAAVTVIEELRPAPAVRSHL

(19) United States
(12) Patent Application Publication
(10) Pub. No.: US 2025/0207108 A1
(43) Pub. Date: Jun. 26, 2025

(54) MONOAMINE OXIDASE AND APPLICATION THEREOF

(71) Applicant: QINGDAO KINGAGROOT CHEMICAL COMPOUND CO., LTD., Qingdao, Shandong (CN)

(72) Inventors: Lei LIAN, Qingdao, Shandong (CN); Xiaoru LI, Qingdao, Shandong (CN); Guizhi LIU, Qingdao, Shandong (CN); Rongbao HUA, Qingdao, Shandong (CN); Sudong MO, Qingdao, Shandong (CN)

(73) Assignee: QINGDAO KINGAGROOT CHEMICAL COMPOUND CO., LTD., Qingdao, Shandong (CN)

(21) Appl. No.: 18/847,224

(22) PCT Filed: Jan. 4, 2023
(86) PCT No.: PCT/CN2023/070432

§ 371 Date: Sep. 14, 2024

(30) Foreign Application Priority Data
Mar. 31, 2022 (CN) 202210332720.2

(51) Int. Cl. C12N 9/06; C12P 17/10

(52) U.S. Cl. CPC C12N 9/0022; C12P 17/10; C12Y 104/03004

(57) ABSTRACT

Provided are monoamine oxidase and an application thereof in a biocatalytic method.

### FIELD

The present invention relates to a monoamine oxidase and use thereof in a biocatalytic method.

### BACKGROUND

The patent application CN102131813A discloses use of a monoamine oxidase in resolving and deracemizing racemic chiral amines via stereospecific oxidation of an enantiomer to the corresponding imine using oxygen. It is nevertheless desirable to provide a new monoamine oxidase useful for the biocatalytic method.

### SUMMARY

This summary is provided to introduce a selection of concepts in a simplified form, and the concepts are further described below in the Detailed Description of the Invention. This summary is not intended to identify key or essential features of the claimed subject matter, nor is it intended to be used to limit the scope of the claimed subject matter. Other features, details, utilities, and advantages of the claimed subject matter will be apparent from the following written Detailed Description of the Invention including those aspects illustrated in the accompanying drawings and defined in the appended claims.

The present invention provides a monoamine oxidase comprising an amino acid sequence having a mutation compared with the amino acid sequence of SEQ ID NO: 1, wherein the mutation is selected from the group consisting of: a mutation of the amino acid at position 63 from phenylalanine into leucine, a mutation of the amino acid at position 65 from threonine into valine, a mutation of the amino acid at position 100 from serine into proline, a mutation of the amino acid at position 141 from threonine into serine, a mutation of the amino acid at position 234 from serine into cysteine, and a combination thereof, in an amino acid sequence corresponding to the amino acid sequence of SEQ ID NO: 1.

In a specific embodiment, the monoamine oxidase comprises an amino acid sequence having a mutation compared with the amino acid sequence of SEQ ID NO: 1, wherein the mutation is selected from the group consisting of:
in an amino acid sequence corresponding to the amino acid sequence of SEQ ID NO: 1, a mutation of the amino acid at position 63 from phenylalanine into leucine and a mutation of the amino acid at position 65 from threonine into valine;
in an amino acid sequence corresponding to the amino acid sequence of SEQ ID NO: 1, a mutation of the amino acid at position 100 from serine into proline and a mutation of the amino acid at position 234 from serine into cysteine;
in an amino acid sequence corresponding to the amino acid sequence of SEQ ID NO: 1, a mutation of the amino acid at position 141 from threonine into serine and a mutation of the amino acid at position 234 from serine into cysteine;
in an amino acid sequence corresponding to the amino acid sequence of SEQ ID NO: 1, a mutation of the amino acid at position 63 from phenylalanine into leucine, a mutation of the amino acid at position 65 from threonine into valine, and a mutation of the amino acid at position 100 from serine into proline;
in an amino acid sequence corresponding to the amino acid sequence of SEQ ID NO: 1, a mutation of the amino acid at position 63 from phenylalanine into leucine, a mutation of the amino acid at position 65 from threonine into valine, and a mutation of the amino acid at position 141 from threonine into serine;
in an amino acid sequence corresponding to the amino acid sequence of SEQ ID NO: 1, a mutation of the amino acid at position 63 from phenylalanine into leucine, a mutation of the amino acid at position 65 from threonine into valine, and a mutation of the amino acid at position 234 from serine into cysteine;
in an amino acid sequence corresponding to the amino acid sequence of SEQ ID NO: 1, a mutation of the amino acid at position 100 from serine into proline, a mutation of the amino acid at position 141 from threonine into serine, and a mutation of the amino acid at position 234 from serine into cysteine;
in an amino acid sequence corresponding to the amino acid sequence of SEQ ID NO: 1, a mutation of the amino acid at position 63 from phenylalanine into leucine, a mutation of the amino acid at position 65 from threonine into valine, a mutation of the amino acid at position 100 from serine into proline, and a mutation of the amino acid at position 141 from threonine into serine;
in an amino acid sequence corresponding to the amino acid sequence of SEQ ID NO: 1, a mutation of the amino acid at position 63 from phenylalanine into leucine, a mutation of the amino acid at position 65 from threonine into valine, a mutation of the amino acid at position 100 from serine into proline, and a mutation of the amino acid at position 234 from serine into cysteine;
in an amino acid sequence corresponding to the amino acid sequence of SEQ ID NO: 1, a mutation of the amino acid at position 63 from phenylalanine into leucine, a mutation of the amino acid at position 65 from threonine into valine, a mutation of the amino acid at position 141 from threonine into serine, and a mutation of the amino acid at position 234 from serine into cysteine; and
in an amino acid sequence corresponding to the amino acid sequence of SEQ ID NO: 1, a mutation of the amino acid at position 63 from phenylalanine into leucine, a mutation of the amino acid at position 65 from threonine into valine, a mutation of the amino acid at position 100 from serine into proline, a mutation of the amino acid at position 141 from threonine into serine, and a mutation of the amino acid at position 234 from serine into cysteine.

In another specific embodiment, the amino acid sequence of the monoamine oxidase further has at least 80%, at least 85%, at least 90%, at least 95%, at least 96%, at least 97%, at least 98%, or at least 99% sequence identity to the amino acid sequence of the monoamine oxidase set forth in SEQ ID NO: 1.

The present invention provides a monoamine oxidase comprising an amino acid sequence having at least 92%, at least 95%, at least 96%, at least 97%, at least 98%, at least 99% or 100% sequence identity to any amino acid sequence selected from the group consisting of SEQ ID NOs: 2-17.

In a specific embodiment, the amino acid sequence of the monoamine oxidase is as set forth in any one of SEQ ID NOs: 2-17.

The present invention further provides a polynucleotide encoding the monoamine oxidase and a host cell comprising the polynucleotide.

The present invention further provides a method for producing a substantially stereomerically pure compound as represented by Formula II or a salt/hydrate thereof, comprising contacting a compound as represented by Formula I with oxygen in the presence of the monoamine oxidase and a cofactor;

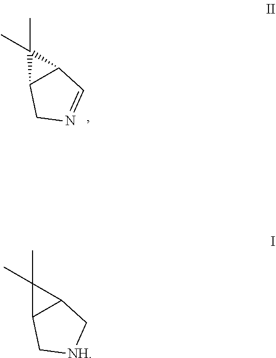

The present invention further provides a method for producing a substantially enantiomerically pure aminosulfonate compound as represented by Formula III or a salt/hydrate thereof, comprising contacting a compound as represented by Formula I with oxygen in the presence of the monoamine oxidase, a cofactor and bisulfite;

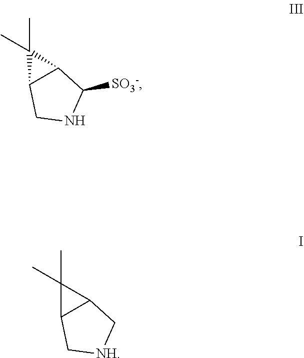

The present invention further provides a method for producing a substantially enantiomerically pure aminonitrile compound as represented by Formula IV or a salt/hydrate thereof, comprising contacting a compound as represented by Formula I with oxygen in the presence of the monoamine oxidase, a cofactor and bisulfite to obtain an aminosulfonate compound, and contacting the aminosulfonate compound with cyanide;

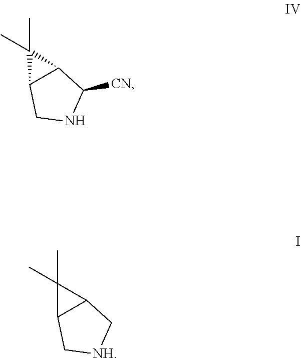

In a specific embodiment, the cofactor is non-covalently associated with a monoamine oxidase.

In another specific embodiment, the cofactor is selected from the group consisting of FAD, FMN, NAD and NADP.

In another specific embodiment, the method further comprises a component catalyzing disproportionation of hydrogen peroxide to molecular oxygen and water; preferably, the component is selected from the group consisting of Pd, Fe and catalase.

The present invention further provides use of the monoamine oxidase in catalyzing oxidation of a compound as represented by Formula I to a substantially stereomerically pure compound as represented by Formula II or a salt/hydrate thereof;

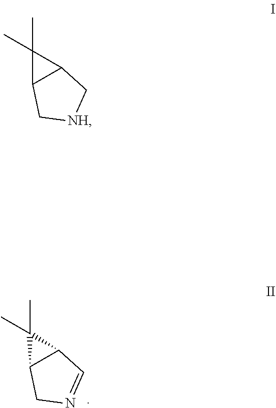

The present invention further provides use of the monoamine oxidase in catalyzing a compound as represented by Formula I to produce a substantially enantiomerically pure aminosulfonate compound as represented by Formula III, an aminonitrile compound as represented by Formula IV, or a salt/hydrate thereof;

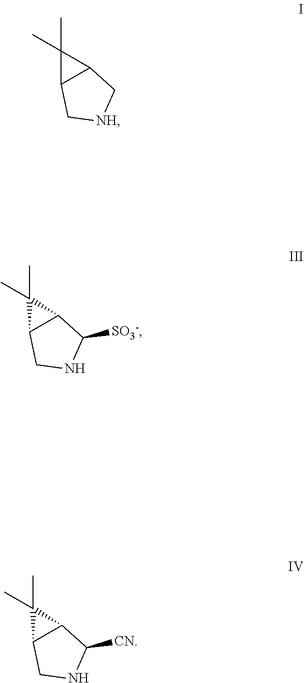

The present invention further provides use of the monoamine oxidase in catalyzing desymmetrization of a compound as represented by Formula I;

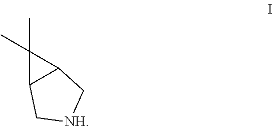

### DETAILED DESCRIPTION OF THE INVENTION

The description set forth below in conjunction with the appended drawings is intended to describe various illustrative embodiments of the disclosed subject matter. Specific features and functionalities are described in conjunction with each illustrative embodiment. However, it will be apparent to one skilled in the art that the disclosed embodiments may be practiced without each of those specific features and functionalities. Moreover, all of the functionalities described in conjunction with an embodiment are intended to be applicable to the additional embodiments described herein except where expressly stated or where the feature or function is incompatible with the additional embodiments. For example, where a given feature or function is expressly described in conjunction with an embodiment but not expressly mentioned in conjunction with an alternative embodiment, it should be understood that the feature or function may be deployed, utilized, or implemented in conjunction with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry, biological emulsion generation, and sequencing technology, which are within the skill of those who practice in the art. Such conventional techniques include polymer array synthesis, hybridization and ligation of polynucleotides, and detection of hybridization using a label. Specific illustrations of suitable techniques can be obtained by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual: Dieffenbach, Dveksler, Eds. (2003), PCR Primer: A Laboratory Manual: Bowtell and Sambrook (2003), DNA Microarrays: A Molecular Cloning Manual: Mount (2004), Bioinformatics: Sequence and Genome Analysis: Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.: Gait, “Oligonucleotide Synthesis: A Practical Approach” 1984, IRL Press, London: Nelson and Cox (2000), Lehninger, Principles of Biochemistry 3^{rd} Ed., W. H. Freeman Pub., New York, N. Y.; and Berg et al. (2002) Biochemistry, 5^{th} Ed., W.H. Freeman Pub., New York, N. Y.: Cell and Tissue Culture: Laboratory Procedures in Biotechnology (Doyle & Griffiths, eds., John Wiley & Sons 1998); Mammalian Chromosome Engineering—Methods and Protocols (G. Hadlaczky, ed., Humana Press 2011); Essential Stem Cell Methods (Lanza and Klimanskaya, eds., Academic Press 2011), all of which are herein incorporated in their entirety by reference for all purposes. Nuclease-specific techniques can be found in, for example, Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes. Basic methods for enzyme engineering may be found in, Enzyme Engineering Methods and Protocols, Samuelson, ed., 2013; Protein Engineering. Kaumaya, ed., (2012); and Kaur and Sharma, “Directed Evolution: An Approach to Engineer Enzymes”, Crit. Rev. Biotechnology, 26:165-69 (2006).

Note that as used herein and in the appended claims, the singular forms “a”, “an”, and “the” include plural referents unless the context clearly dictates otherwise. Thus, for example, reference to “an oligonucleotide” refers to one or more oligonucleotides, and reference to “an automated system” includes reference to equivalent steps and methods for use with the system known to one skilled in the art, and so forth. Additionally, it is to be understood that terms such as “left.” “right”, “top”, “bottom”, “front”, “rear”, “side”, “height”, “length”, “width”, “upper”, “lower”, “interior”, “exterior”, “inner”, “outer” and the like that may be used herein merely describe points of reference and do not necessarily limit embodiments of the present disclosure to any particular orientation or configuration. Furthermore, terms such as “first”, “second”, “third”, etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which the present invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the present invention. The upper and lower limits of these smaller ranges may independently be included in the smaller ranges, and are also encompassed within the present invention, subject to any specifically excluded limit in the stated range. Where the stated range includes one or both of the limits, ranges excluding any one or both of those included limits are also included in the present invention.

In the following description, numerous specific details are set forth to provide a more thorough understanding of the present invention. However, it will be apparent to one of ordinary skill in the art that the present invention may be practiced without one or more of these specific details. In other instances, features and procedures well known to those skilled in the art have not been described in order to avoid obscuring the present invention.

The monoamine oxidase of the present disclosure that enables the oxidation of the amine compound of Formula I to the corresponding imine compound of Formula II has one or more amino acid substitutions compared to the amino acid sequence of SEQ ID NO: 1. Such amino acid substitutions provide the monoamine oxidase with one or more improved properties including increased enzymatic activity, increased stereospecificity, increased thermostability, increased solvent stability, reduced product inhibition, reduced substrate inhibition, or reduced sensitivity to reaction co-products. Such amino acid substitutions may also improve the expression level, solubility, and/or stability of the monoamine oxidase in a host cell, e.g., as a recombinantly-expressed protein in a heterologous host cell, such as but not limited to an E. coli host cell.

The present disclosure further provides a polynucleotide encoding the monoamine oxidase and a method for using the polypeptide in a biocatalytic method disclosed.

As used herein, the following terms are intended to have the following meanings.

“Monoamine oxidase” refers to a polypeptide having an enzymatic capability of oxidizing a compound of the foregoing structural Formula I to the corresponding product of the foregoing structural Formula II. The polypeptide typically utilizes an oxidized cofactor, such as but not limited to flavin adenine dinucleotide (FAD), flavin adenine mononucleotide (FMN), nicotinamide adenine dinucleotide (NAD), or nicotinamide adenine dinucleotide phosphate (NADP). In a specific embodiment, the oxidized cofactor is FAD. The monoamine oxidase as used herein includes a naturally occurring (wild-type) monoamine oxidase as well as a non-naturally occurring engineered polypeptide generated by human manipulation.

“Coding sequence” refers to a portion of a nucleic acid (e.g., a gene) that encodes an amino acid sequence of a protein.

“Naturally-occurring” or “wild-type” refers to the form found in nature. For example, a naturally occurring or wild-type polypeptide or polynucleotide sequence is a sequence present in an organism that can be isolated from a source in nature and has not been intentionally modified by human manipulation.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, but are not limited to, recombinant cells expressing genes that are not found within the native (non-recombinant) form of the cell or expressing native genes that are otherwise expressed at a different level.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise addition(s) or deletion(s) (i.e., gap(s)) as compared to the reference sequence (which does not comprise addition(s) or deletion(s)) for optimal alignment of the two sequences. The percentage may be calculated by determining the number of positions at which the identical nucleic acid base or amino acid residue occurs in both sequences to yield the number of matched positions, dividing the number of matched positions by the total number of positions in the comparison window and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the comparison window and multiplying the result by 100 to yield the percentage of sequence identity: One skilled in the art appreciates that there are many established algorithms available to align two sequences. Optimal alignment of sequences for comparison can be conducted, e.g., by the local homology algorithm of Smith and Waterman, 1981, Adv. Appl. Math. 2:482; by the homology alignment algorithm of Needleman and Wunsch, 1970, J. Mol. Biol. 48:443; by the search for similarity method of Pearson and Lipman, 1988, Proc. Natl. Acad. Sci. USA 85:2444: by computerized implementations of these algorithms (GAP, BESTFIT, FASTA, and TFASTA in the GCG Wisconsin Software Package), or by visual inspection (see generally; Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining the percentage of sequence identity and sequence similarity are the BLAST and BLAST 2.0 algorithms, which are described in Altschul et al., 1990, J. Mol. Biol. 215:403-410 and Altschul et al., 1977, Nucleic Acids Res. 3389-3402, respectively. Software for performing BLAST analysis is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, parameters M (reward score for a pair of matching residues; always>0) and N (penalty score for mismatching residues; always<0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. Parameters W. T, and X of the BLAST algorithm determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys. Madison Wis.), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides over a “comparison window” to identify and compare sequence similarity of local regions.

“Comparison window” refers to a conceptual segment of at least about 20 contiguous nucleotide positions or amino acid residues wherein a sequence may be compared to a reference sequence of at least 20 contiguous nucleotides or amino acids and wherein the portion of the sequence in the comparison window may comprise additions or deletions (i.e., gaps) of 20% or less as compared to the reference sequence (which does not comprise additions or deletions) for optimal alignment of the two sequences. The comparison window can be longer than 20 contiguous residues, and includes, optionally 30 contiguous residues, 40 contiguous residues, 50 contiguous residues, 100 contiguous residues, or longer windows.

“Substantial identity” refers to a polynucleotide or polypeptide sequence that has at least 80% sequence identity, at least 85% identity and 89% to 95% sequence identity, more usually at least 99% sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which in total are 20% or less of the reference sequence over the comparison window. In specific embodiments applied to polypeptides, the term “substantial identity” means that two polypeptide sequences, when optimally aligned, such as by the programs GAP or BESTFIT using default gap weights, share at least 80% sequence identity, preferably at least 89% sequence identity, at least 95% sequence identity or more (e.g., 99% sequence identity). Preferably, residue positions which are not identical differ by conservative amino acid substitutions.

“Corresponding to”, “reference to”, or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered monoamine oxidase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (e.e.) calculated therefrom according to the formula [Major enantiomer−Minor enantiomer]/[Major enantiomer+Minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastercometric excess (d.e.). Enantiomeric excess and diastereometric excess are types of stereometric excess.

“High stereoselectivity” refers to a monoamine oxidase polypeptide that is capable of converting a substrate to the corresponding product with at least about 99% stereometric excess.

“Stereospecificity” refers to the preferential conversion in a chemical or enzymatic reaction of one stereoisomer over another. Stereospecificity can be partial, where the conversion of one stereoisomer is favored over the other, or it may be complete where only one stereoisomer is converted.

“Chemoselectivity” refers to the preferential formation in a chemical or enzymatic reaction of one product over another.

“Improved enzyme property.” refers to a monoamine oxidase polypeptide that exhibits an improvement in any enzyme property as compared to a reference monoamine oxidase. For the engineered monoamine oxidase polypeptides described herein, the comparison is generally made to the wild-type monoamine oxidase, although in some embodiments the reference monoamine oxidase can be another improved engineered monoamine oxidase. Enzyme properties for which improvement is desirable include, but are not limited to, enzymatic activity (which can be expressed in terms of percent conversion of the substrate), thermostability, pH activity profile, cofactor requirements, refractoriness to inhibitors (e.g., product inhibition), stereospecificity, stereoselectivity (including enantioselectivity), solubility; stability and expression level in a host cell.

“Increased enzymatic activity” refers to an improved property of the engineered monoamine oxidase polypeptides, which can be represented by an increase in specific activity (e.g., product produced/time/weight protein) or an increase in percent conversion of the substrate to the product (e.g., percent conversion of starting amount of substrate to product in a specified time period using a specified amount of monoamine oxidase) as compared to the reference monoamine oxidase. Exemplary methods to determine enzymatic activity are provided in the Examples. Any property relating to enzymatic activity may be affected, including the classical enzyme properties of Kₘ, Vₘₐₓ or k_{cat}, changes of which can lead to increased enzymatic activity. Improvements in enzymatic activity can be from about 1.5 times the enzymatic activity of the corresponding wild-type monoamine oxidase, to as much as 2 times, 5 times, 10 times, 20 times, 25 times, 50 times, 75 times, 100 times or higher enzymatic activity than the naturally occurring monoamine oxidase or another engineered monoamine oxidase from which the monoamine oxidase polypeptides were derived. It is understood by one skilled in the art that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 10⁸ to 10⁹ (M⁻¹ s⁻¹). Hence, any improvements in the enzymatic activity of the monoamine oxidase will have an upper limit related to the diffusion rate of the substrates acted on by the monoamine oxidase. Monoamine oxidase activity can be measured using published methods, or adapted methods thereof, for measuring monoamine oxidase, such as, but not limited to those disclosed by Zhou et al. (Zhou et al. “A One-Step Fluorometric Method for the Continuous Measurement of Monoamine Oxidase Activity,” 1997 Anal. Biochem. 253:169-74) and Szutowicz et al. (Szutowicz et al., “Colorimetric Assay for Monoamine Oxidase in Tissues Using Peroxidase and 2,2′-Azino(3-ethylbenzthaizoline-6-sulfonic Acid) as Chromogen,” 1984, Anal. Biochem. 138:86-94). Comparisons of enzymatic activities are made using a defined preparation of enzyme, a defined assay under a set condition, and one or more defined substrates, as further described in detail herein or using the methods of, e.g., Zhou and Szutowicz. Generally, when lysates are compared, the numbers of cells and the amount of protein assayed are determined as well identical expression systems and identical host cells being used to minimize variations in amount of enzyme produced by the host cells and present in the lysates.

“Conversion”: refers to the enzymatic oxidation of a substrate to the corresponding product. “Percent conversion” refers to the percent of the substrate that is oxidized to the product within a period of time under specified conditions. Thus, the “enzymatic activity” or “activity” of a monoamine oxidase polypeptide can be expressed as “percent conversion” of the substrate to the product.

“Thermostable” refers to a monoamine oxidase polypeptide that maintains similar activity (e.g., more than 60% to 80%) after exposure to elevated temperatures (e.g., 40-80° C.) for a period of time (e.g., 0.5-24 hrs) compared to the untreated enzyme.

“Solvent stable” refers to a monoamine oxidase polypeptide that maintains similar activity (e.g., more than 60% to 80%) after exposure to varying concentrations (e.g., 5-99%) of solvent (isopropanol, tetrahydrofuran, 2-methyltetrahydrofuran, acetone, toluene, butylacetate, methyl tert-butylether, etc.) for a period of time (e.g., 0.5-24 hrs) compared to the untreated enzyme.

“pH stable” refers to a monoamine oxidase polypeptide that maintains similar activity (e.g., more than 60% to 80%) after exposure to high or low pH (e.g., 4.5-6 or 8-12) for a period of time (e.g., 0.5-24 hrs) compared to the untreated enzyme.

“Thermo- and solvent stable” refers to a monoamine oxidase polypeptide that is both thermostable and solvent stable.

“Hydrophilic amino acid or residue” refers to an amino acid or residue having a side chain exhibiting a hydrophobicity of less than zero according to the normalized consensus hydrophobicity scale of Eisenberg et al., 1984, J. Mol. Biol. 179:125-142. Genetically encoded hydrophilic amino acids include L-Thr (T), L-Ser(S), L-His (H), L-Glu (E), L-Asn (N), L-Gln (Q), L-Asp (D), L-Lys (K) and L-Arg (R).

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic amino acid or residue” refers to a hydrophilic amino acid or residue having a side chain exhibiting a pK value of greater than about 6 when the amino acid is included in a peptide or polypeptide. Basic amino acids typically have positively charged side chains at physiological pH due to association with a hydronium ion. Genetically encoded basic amino acids include L-Arg (R) and L-Lys (K).

“Polar amino acid or residue” refers to a hydrophilic amino acid or residue having a side chain that is uncharged at physiological pH but has at least one bond in which the pair of electrons shared in common by two atoms is held more closely by one of the atoms. Genetically encoded polar amino acids include L-Asn (N), L-Gln (Q), L-Ser(S) and L-Thr (T).

“Hydrophobic amino acid or residue” refers to an amino acid or residue having a side chain exhibiting a hydrophobicity of greater than zero according to the normalized consensus hydrophobicity scale of Eisenberg et al., 1984, J. Mol. Biol. 179:125-142. Genetically encoded hydrophobic amino acids include L-Pro (P), L-Ile (I), L-Phe (F), L-Val (V), L-Leu (L), L-Trp (W), L-Met (M), L-Ala (A) and L-Tyr (Y).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y) and L-Trp (W). Although L-His (H) is sometimes classified as a basic residue due to the pKa of its heteroaromatic nitrogen atom, or as an aromatic residue as its side chain includes a heteroaromatic ring, herein histidine is classified as a hydrophilic residue or as a “constrained residue” (see below).

“Constrained amino acid or residue” refers to an amino acid or residue that has a constrained geometry: Herein, constrained residues include L-pro (P) and L-his (H). Histidine has a constrained geometry because it has a relatively small imidazole ring. Proline has a constrained geometry because it also has a five-membered ring.

“Non-polar amino acid or residue” refers to a hydrophobic amino acid or residue having a side chain that is uncharged at physiological pH and has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M) and L-Ala (A).

“Aliphatic amino acid or residue” refers to a hydrophobic amino acid or residue having an aliphatic hydrocarbon side chain. Genetically encoded aliphatic amino acids include L-Ala (A), L-Val (V), L-Leu (L) and L-Ile (I).

“Cysteine”: The amino acid L-Cys (C) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges. The ability of L-Cys (C) (and other amino acids with —SH-containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure L-Cys (C) is categorized into its own unique group.

“Small amino acid or residue” refers to an amino acid or residue having a side chain that is composed of a total three or fewer carbon and/or heteroatoms (excluding α-carbon and hydrogen). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser(S), L-Thr (T) and L-Asp (D).

“Hydroxyl-containing amino acid or residue” refers to an amino acid containing a hydroxyl (—OH) moiety: Genetically-encoded hydroxyl-containing amino acids include L-Ser(S), L-Thr (T) and L-Tyr (Y).

“Conservative” amino acid substitutions or mutations refer to the interchangeability of residues having similar side chains, and thus typically involve substitution of an amino acid in a polypeptide with an amino acid belonging to the same or similar defined class of amino acids. However, as used herein, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. Table 1 below shows exemplary conservative substitutions.

TABLE 1
Conservative substitutions

| Residue | Possible conservative mutation |
|---|---|
| A, L, V, I | Other aliphatic residues (A, L, V, I) |
| | Other non-polar residues (A, L, V, I, G, M) |
| G, M | Other non-polar residues (A, L, V, I, G, M) |
| D, E | Other acidic residues (D, E) |
| K, R | Other basic residues (K, R) |
| P, H | Other constrained residues (P, H) |
| N, Q, S, T | Other polar residues (N, Q, S, T) |
| Y, W, F | Other aromatic residues (Y, W, F) |
| C | None |

“Non-conservative substitution” refers to substitution or mutation of an amino acid in a polypeptide with an amino acid having significantly differing side chain properties. Non-conservative substitutions may use amino acids between, rather than within, the defined groups listed above. In an embodiment, a non-conservative mutation affects (a) the structure of the peptide backbone in the area of the substitution (e.g., proline substitutes glycine) (b) the charge or hydrophobicity, or (c) the bulk of the side chain.

“Deletion” refers to modification to a polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids or up to 20% of the total number of amino acids making up the reference enzyme while retaining enzymatic activity and/or retaining the improved properties of an engineered monoamine oxidase. Deletions can be directed to the internal portions and/or terminal portions of the polypeptide. In various embodiments, the deletion can comprise a continuous segment or can be discontinuous.

“Insertion” refers to modification to a polypeptide by addition of one or more amino acids to the reference polypeptide. In some embodiments, the improved engineered monoamine oxidases comprise insertions of one or more amino acids to the naturally occurring monoamine oxidase as well as insertions of one or more amino acids to other improved monoamine oxidase polypeptides. Insertions can be in the internal portions of the polypeptide, or to the carboxyl or amino terminus. Insertions as used herein include fusion proteins as known in the art. The insertion can be a contiguous segment of amino acids or separated by one or more of the amino acids in the naturally occurring polypeptide.

“Different from” or “differs from” with respect to a designated reference sequence refers to difference of a given amino acid or polynucleotide sequence when aligned to the reference sequence. Generally, the differences can be determined when the two sequences are optimally aligned. Differences include insertions, deletions, or substitutions of amino acid residues in comparison to the reference sequence.

“Fragment” as used herein refers to a polypeptide that has an amino-terminal and/or carboxyl-terminal deletion, but where the remaining amino acid sequence is identical to the corresponding positions in the sequence. Fragments can be at least 14 amino acids long, at least 20 amino acids long, at least 50 amino acids long or longer, and up to 70%, 80%, 90%, 95%, 98%, or 99% of the full-length monoamine oxidase polypeptide.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., a host cell or in vitro synthesis). The improved monoamine oxidase may be present within a cell, present in a cellular medium, or prepared in various forms, such as lysates or isolated preparations. As such, in some embodiments, the improved monoamine oxidase can be an isolated polypeptide.

“Substantially pure polypeptide” refers to a composition in which the polypeptide substance is the predominant substance present (i.e., on a molar or weight basis it is more abundant than any other individual macromolecular substance in the composition) and is generally a substantially purified composition when the object substance comprises at least about 50% of the macromolecular substances present by mole or % weight. Generally, a substantially pure monoamine oxidase composition will comprise about 60% or more, about 70% or more, about 80% or more, about 90% or more, about 95% or more, or about 98% or more of all macromolecular substances by mole or % weight present in the composition. In some embodiments, the object substance is purified to essential homogeneity (i.e., contaminant substances cannot be detected in the composition by conventional detection methods) wherein the composition consists essentially of a single macromolecular substance. Solvent substances, small molecules (<500 Daltons), and elemental ion substances are not considered macromolecular substances. In some embodiments, the isolated improved monoamine oxidase polypeptide is a substantially pure polypeptide composition.

“Stringent hybridization” used herein refers to conditions under which nucleic acid hybrids are stable. As known to one skilled in the art, the stability of hybrids is reflected in the melting temperature (Tₘ) of the hybrids. In general, the stability of a hybrid is a function of ion strength, temperature, G/C content, and the presence of chaotropic agents. The Tₘ values for polynucleotides can be calculated using known methods for predicting melting temperatures (see, e.g., Baldino et al., Methods Enzymology 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res 18:6409-6412 (erratum, 1991, Nucleic Acids Res 19:698); Sambrook et al., supra; Suggs et al., 1981, In Developmental Biology Using Purified Genes (Brown et al., eds.), pp. 683-693, Academic Press; and Wetmur, 1991. Crit. Rev Biochem Mol Biol 26:227-259. All publications were incorporated herein by reference). In some embodiments, the polynucleotide encodes the polypeptide disclosed herein and hybridizes under defined conditions, such as moderately stringent or highly stringent conditions, with the complementary sequence of the sequence encoding the engineered monoamine oxidase of the present disclosure.

“Hybridization stringency” relates to such washing conditions of nucleic acids. Generally, hybridization reactions are performed under conditions of low stringency, followed by washes with varying but high stringency: The term “moderately stringent hybridization” refers to conditions that permit target DNA to bind a complementary nucleic acid that has about 60% identity, preferably about 75% identity, or about 85% identity to the target DNA, and has greater than about 90% identity to the target polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS at 42° C. “Highly stringent hybridization” refers generally to conditions that are about 10° C. or less lower from the thermal melting temperature Tₘ as determined under the solution condition for a defined polynucleotide sequence. In some embodiments, a highly stringent condition refers to conditions that permit hybridization of only those nucleic acid sequences that form stable hybrids in 0.018 M NaCl at 65° C. (i.e., if a hybrid is not stable in 0.018 M NaCl at 65° C., it will not be stable under highly stringent conditions, as contemplated herein). Highly stringent conditions can be provided, for example, by hybridization in conditions equivalent to 50% formamide, 5×Denhart's solution, 5×SSPE, 0.2% SDS at 42° C., followed by washing in 0.1×SSPE and 0.1% SDS at 65° C. Other highly stringent hybridization conditions, as well as moderately stringent conditions, are described in the references cited above.

“Heterologous” polynucleotide refers to a polynucleotide that is introduced into a host cell by laboratory techniques and includes polynucleotides that are removed from a host cell, subjected to laboratory manipulation, and then reintroduced into the host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus proteins with low copy number, and the aggregate protein coding regions of an organism's genome. In some embodiments, the polynucleotides encoding the monoamine oxidases may be subjected to codon optimization for optimal production from the host organism selected for expression.

“Preferred, optimal, high codon usage bias codons” refers interchangeably to codons that are used at higher frequency in the protein coding regions than other codons that code for the same amino acid. The preferred codons may be determined in relation to codon usage in a single gene, codon usage in a set of genes of common function or origin, codon usage in highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package: Codon W. John Peden, University of Nottingham: McInerney, J. O, 1998, Bioinformatics 14:372-73: Stenico ct al., 1994, Nucleic Acids Rcs. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066). The data source for obtaining codon usage may rely on any available nucleotide sequence capable of coding for a protein. These data sets include nucleic acid sequences actually known to encode expressed proteins (e.g., complete protein coding sequences—CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281: Tiwari et al., 1997, Comput. Appl. Biosci. 13:263-270).

“Control sequence” is defined herein to include all components, which are necessary or advantageous for the expression of a polypeptide of the present disclosure. Each control sequence may be native or foreign to the nucleic acid sequence encoding the polypeptide. Such control sequences include, but are not limited to, a leader sequence, polyadenylation sequence, propeptide sequence, promoter, signal peptide sequence, and transcription terminator. At a minimum, the control sequences include a promoter, and transcriptional and translational termination signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed at a position relative to the coding sequence of the DNA sequence such that the control sequence directs the expression of a polynucleotide and/or polypeptide.

“Promoter sequence” is a nucleic acid sequence that is recognized by a host cell for expression of the coding region. A control sequence may comprise an appropriate promoter sequence. The promoter sequence contains transcriptional control sequences, which mediate the expression of the polypeptide. The promoter may be any nucleic acid sequence which shows transcriptional activity in the host cell of choice, including mutant, truncated, and hybrid promoters, and may be obtained from genes encoding extracellular or intracellular polypeptides either homologous or heterologous to the host cell.

As used herein, the terms “stereoisomer”, “stereoisomeric form” and the like are general terms for all isomers of individual molecules that differ only in the orientation of their atoms in space. It includes enantiomers and isomers of compounds with more than one chiral center that are not mirror images of one another (“diastereomers”).

The term “chiral center” refers to a carbon atom to which four different groups are attached.

The term “enantiomer” or “enantiomeric” refers to a molecule that is non-superimposeable on its mirror image and hence optically active where the enantiomer rotates the plane of polarized light in one direction and its mirror image rotates the plane of polarized light in the opposite direction.

The term “racemic” refers to a mixture of equal parts of enantiomers, which is optically inactive.

The term “resolution” refers to the separation or concentration or depletion of one of the two enantiomeric forms of a molecule.

“Substantially enantiomerically pure” as used herein means that the indicated enantiomer of a compound is present to a greater extent or degree than another enantiomer of the same compound. Accordingly, in particular embodiments, a substantially enantiomerically pure compound is present in 80%, 85%, 90%, 91%, 92%, 93%, 94%, 95%, 96%, 97%, 98%, or 99% enantiomeric excess over another enantiomer of the same compound.

“Substantially stereometrically pure” as used herein means that the indicated enantiomer or diastereomer of a compound is present to a greater extent or degree than another enantiomer or diastereomer of the same compound. As noted above with respect to “stereoselectivity”, enantiomeric excess and diastercometric excess are types of stereometric excess. Accordingly, in particular embodiments, a substantially stereomerically pure compound is present in 80%, 85%, 90%, 91%, 92%, 93%, 94%, 95%, 96%, 97%, 98%, or 99% stereometric excess over another enantiomer or diastereomer of the same compound.

As will be appreciated by one skilled in the art, the polypeptides described herein are not restricted to the genetically encoded amino acids. In addition to the genetically encoded amino acids, the polypeptides described herein may comprise, either in whole or in part, naturally-occurring and/or synthetic non-encoded amino acids. Certain commonly encountered non-encoded amino acids which the monoamine oxidases described herein may comprise include, but are not limited to: the D-stereoisomers of the genetically-encoded amino acids: 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); 1-butylalanine (Bua); 1-butylglycine (Bug); N-methylisoleucine (Melle); phenylglycine (Phg); cyclohexylalanine (Cha); norleucine (Nle); naphthylalanine (Nal); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (lnAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillaminc (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhc); phosphoserine (pSer); phosphothreonine (pThr); homoaspartic acid (hAsp); homoglutanic acid (hGlu); 1-aminocyclopent-(2 or 3)-ene-4 carboxylic acid: pipecolic acid (PA), azetidine-3-carboxylic acid (ACA); 1-aminocyclopentane-3-carboxylic acid: allylglycine (aOly); propargylglycine (pgGly); homoalanine (hAla); norvaline (n Val); homoleucine (hLeu), homovaline (h Val); homoisolencine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids that the monoamine oxidases described herein may comprise will be apparent to one skilled in the art (see, e.g., the various amino acids provided in Fasman, 1989, CRC Practical Handbook of Biochemistry and Molecular Biology, CRC Press, Boca Raton, Fla., at pp. 3-70 and the references cited therein, all of which are incorporated by reference). These amino acids may be in either the L- or D-configuration.

One skilled in the art will recognize that the monoamine oxidases disclosed herein may also comprise amino acids or residues bearing side chain protecting groups. Non-limiting examples of such protected amino acids, which in this case belong to the aromatic category, include (protecting groups listed in parentheses), but are not limited to: Arg (tos), Cys (methylbenzyl), Cys (nitropyridinesulfenyl), Glu (δ-benzylester), Gln (xanthyl), Asn (N-δ-xanthyl), His (bom), His (bnezyl), His (tos), Lys (fmoc), Lys (tos), Ser (O-benzyl), Thr (O-benzyl) and Tyr (O-benzyl).

Non-encoded amino acids that are conformationally constrained that the monoamine oxidases described herein may comprise include, but are not limited to, N-methyl amino acids (L-configuration); 1-aminocyclopent-(2 or 3)-ene-4-carboxylic acid; pipecolic acid; azetidine-3-carboxylic acid: homoproline (hPro); and 1-aminocyclopentane-3-carboxylic acid.

As described above, the various modifications introduced into the naturally occurring polypeptide to generate an engineered monoamine oxidase can be targeted to a specific enzyme property.

In another aspect, the present disclosure provides polynucleotides encoding the engineered monoamine oxidases disclosed herein. The polynucleotides may be operably linked to one or more heterologous regulatory sequences that control gene expression to create a recombinant polynucleotide capable of expressing the polypeptide. Expression constructs containing a heterologous polynucleotide encoding the engineered monoamine oxidase can be introduced into appropriate host cells to express the corresponding monoamine oxidase polypeptide.

Because of the knowledge of the codons corresponding to the various amino acids, availability of a protein sequence provides a description of all the polynucleotides capable of encoding the subject. The degeneracy of the genetic code where the same amino acids are encoded by alternative or synonymous codons allows an extremely large number of nucleic acids to be made, all of which encode the improved monoamine oxidases disclosed herein. Thus, having identified a particular amino acid sequence, one skilled in the art could make any number of different nucleic acids by simply modifying the sequence of one or more codons in a way which does not change the amino acid sequence of the protein.

In some embodiments, the polynucleotide comprises a nucleotide sequence encoding a monoamine oxidase with an amino acid sequence that has at least about 80% or more sequence identity, about 85% or more sequence identity; about 90% or more sequence identity, about 95% or more sequence identity; about 96% or more sequence identity, about 97% or more sequence identity, about 98% or more sequence identity, or 99% or more sequence identity to any of the reference engineered monoamine oxidase described herein.

In various embodiments, the codons are preferably selected to fit the host cell in which the protein is being produced. For example, preferred codons used in bacteria are used to express the gene in bacteria: preferred codons used in yeast are used for expression in yeast; and preferred codons used in mammals are used for expression in mammalian cells.

In certain embodiments, not all codons need to be replaced to optimize the codon usage of the monoamine oxidase since the natural sequence will comprise preferred codons and because use of preferred codons may not be required for all amino acid residucs. Consequently, codon-optimized polynucleotides encoding the monoamine oxidases may contain preferred codons at about 40%, 50%, 60%, 70%, 80%, or greater than 90% of codon positions of the full-length coding region.

In other embodiments, the polynucleotides comprise polynucleotides that encode the monoamine oxidases described herein and have about 80% or more sequence identity, about 85% or more sequence identity; about 90% or more sequence identity; about 95% or more sequence identity, about 98% or more sequence identity, or 99% or more sequence identity at the nucleotide level to a reference polynucleotide encoding an engineered monoamine oxidase.

An isolated polynucleotide encoding an improved monoamine oxidase may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, Molecular Cloning: A Laboratory Manual, 3^{rd} Ed., Cold Spring Harbor Laboratory Press; and Current Protocols in Molecular Biology, Ausubel. F. ed., Greene Pub. Associates, 1998, updated to 2006.

For bacterial host cells, suitable promoters for directing the transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (VIIIa-Kamaroff et al., 1978, Proc. Natl. Acad. Sci. USA 75:3727-3731), as well as the tac promoter (DeBoer et al., 1983, Proc. Natl. Acad. Sci. USA 80:21-25). Further promoters are described in “Useful proteins from recombinant bacteria” in Scientific American, 1980, 242:74-94; and in Sambrook et al., supra.

For filamentous fungal host cells, suitable promoters for directing the transcription of the nucleic acid constructs of the present disclosure include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline proteinase, Aspergillus oryzae triose phosphate isomerase, Aspergillus nidulans acetamidase, and Fusarium oxysporum trypsin-like proteinase (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters can be obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8:423-488.

The control sequence may also be a suitable transcription terminator sequence, which is a sequence recognized by a host cell to terminate transcription. The terminator sequence is operably linked to the 3′-terminus of the nucleic acid sequence encoding the polypeptide. Any terminator which is functional in the host cell of choice may be used in the methods disclosed herein.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like proteinase.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, which is a nontranslated region of an mRNA that is important for translation by the host cell. The leader sequence is operably linked to the 5′-terminus of the nucleic acid sequence encoding the polypeptide. Any leader sequence that is functional in the host cell of choice may be used. Exemplary leader sequences for filamentous fungal host cells are obtained from the genes for Aspergillus oryzae TAKA amylase and Aspergillus nidulans triose phosphate isomerase. Suitable leader sequences for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the nucleic acid sequence and which, when transcribed, is recognized by the host cell as a signal to add polyadenosine residues to transcribed mRNA. Any polyadenylation sequence which is functional in the host cell of choice may be used in the methods disclosed herein. Exemplary polyadenylation sequences for filamentous fungal host cells can be obtained from the genes for Aspergillus oryzae TAKA amylase, Aspergillus niger glucoamylase, Aspergillus nidulans anthranilate synthase, Fusarium oxysporum trypsin-like proteinase, and Aspergillus niger alpha-glucosidase. Useful polyadenylation sequences for yeast host cells are described by Guo and Sherman, 1995, Mol Cell Bio 15:5983-5990.

The control sequence may also be a signal peptide coding region that encodes an amino acid sequence linked to the amino terminus of a polypeptide and directs the encoded polypeptide into the secretory pathway of a cell. The 5′ end of the coding sequence of the nucleic acid sequence may inherently contain a signal peptide coding region naturally linked in the translation reading frame with the fragment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region.

Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the methods disclosed herein.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteinases (nprT, nprS, nprM), and Bacillus subtilis prsA. Further signal peptides are described by Simonen and Palva, 1993, Microbiol Rev 57:109-137.

Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Signal peptides useful for yeast host cells can be obtained from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that encodes an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline proteinase (aprE), Bacillus subtilis neutral proteinase (nprT), Saccharomyces cerevisiae alpha-factor, Rhizomucor miehei aspartic proteinase, and Myceliophthora thermophila lactase (WO 95/33836).

In the case that both signal peptide and propeptide regions are present at the amino terminus of a polypeptide, the propeptide region is positioned next to the amino terminus of the polypeptide and the signal peptide region is positioned next to the amino terminus of the propeptide region.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter.

Other examples of regulatory sequences are those which allow for gene amplification. In eukaryotic systems, these regulatory sequences include the dihydrofolate reductase gene, which is amplified in the presence of methotrexate, and the metallothionein genes, which are amplified with heavy metals. In these cases, the nucleic acid sequence encoding the monoamine oxidase of the present disclosure would be operably linked with the regulatory sequence.

Thus, in another embodiment, the present disclosure is also directed to a recombinant expression vector comprising a polynucleotide encoding an engineered monoamine oxidase or a variant thereof, and one or more expression regulating regions such as a promoter, a terminator, and a replication origin, depending on the type of hosts into which they are to be introduced. The various nucleic acids and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present disclosure may be expressed by inserting the nucleic acid sequence of the present disclosure or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector typically depends on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector of the present disclosure preferably contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes obtained from Bacillus subtilis or Bacillus licheniformis, or markers, which confer antibiotic resistance such as ampicillin, kanamycin, chloramphenicol or tetracycline resistance. Suitable markers for yeast host cells are ADE2, HIS3, LEU2, LYS2, MET3, TRP1, and URA3.

Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), s ((sulfate adenyltransferase), and trp ((anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

The expression vectors of the present disclosure preferably contain an element(s) that permits integration of the vector into the host cell's genome or permits autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise a replication origin enabling the vector to replicate autonomously in the host cell in question. Examples of bacterial replication origins include P15A ori or the replication origins of plasmids pBR322, pUC19, pACYC177 (which plasmid has the P15A ori), or pACYC184 permitting replication in E. coli, and pUB110, pE194, pTA1060, or pAM.beta. 1 permitting replication in Bacillus. Examples of replication origins for use in a yeast host cell include the 2-micron replication origin, ARS1, ARS4, the combination of ARS1 and CEN3, and the combination of ARS4 and CEN6. The replication origin may be one comprising a mutation which makes its function temperature-sensitive in the host cell (see, e.g., Ehrlich, 1978, Proc Natl Acad. Sci. USA 75:1433).

More than one copy of the nucleic acid sequence of the present disclosure may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by culturing the cells in the presence of an appropriate selectable agent.

Many of the expression vectors for use in the methods disclosed herein are commercially available. Suitable commercial expression vectors include p3×FLAG™™ expression vectors from Sigma-Aldrich Chemicals, St. Louis Mo., which comprises a CMV promoter, hGH polyadenylation site for expression in mammalian host cells, a pBR322 replication origin and ampicillin resistance markers for amplification in E. coli. Other suitable expression vectors include pBluescriptll SK(−) and pBK-CMV, which are commercially available from Stratagene, LaJolla Calif., and plasmids which are derived from pBR322 (Gibco BRL), pUC (Gibco BRL), pREP4, pCEP4 (Invitrogen) or pPoly (Lathe et al., 1987, Gene 57:193-201).

In another aspect, the present disclosure provides a host cell comprising a polynucleotide encoding the improved monoamine oxidase of the present disclosure, wherein the polynucleotide is operably linked to one or more control sequences for expression of the monoamine oxidase in the host cell. Host cells for use in expressing the monoamine oxidase polypeptides encoded by the expression vectors of the present disclosure are well known in the art and include but are not limited to, bacterial cells, such as E. coli, Lactobacillus kefir, Lactobacillus brevis, Lactobacillus minor, Streptomyces and Salmonella typhimurium cells: fungal cells, such as yeast cells (e.g., Saccharomyces cerevisiae or Pichia pastoris (ATCC Accession No. 201178)); insect cells such as Drosophila S2 and Spodoptera Sf9 cells: animal cells such as CHO, COS, BHK, 293, and Bowes melanoma cells; and plant cells. Appropriate culture mediums and growth conditions for the above-described host cells are well known in the art.

Polynucleotides for expression of the monoamine oxidase may be introduced into cells by various methods known in the art. Techniques include but are not limited to electroporation, biolistic particle bombardment, liposome-mediated transfection, calcium chloride transfection, and protoplast fusion. Various methods for introducing polynucleotides into cells will be apparent to one skilled in the art.

An exemplary host cell is Escherichia coli W3110. An expression vector was created by operably linking a polynucleotide encoding an improved monoamine oxidase into the plasmid pCK110900 operably linked to the lac promoter under control of the lacl repressor. The expression vector also contained the P15a replication origin and the chloramphenicol resistance gene. Cells containing the subject polynucleotide in Escherichia coli W3110 were isolated by subjecting the cells to chloramphenicol selection.

The engineered monoamine oxidases can be obtained by subjecting the polynucleotide encoding the naturally occurring monoamine oxidase to mutagenesis and/or directed evolution methods. An exemplary directed evolution technique is mutagenesis and/or DNA shuffling as described in Stemmer, 1994, Proc Natl Acad Sci USA 91:10747-10751; WO 95/22625; WO 97/0078; WO 97/35966; WO 98/27230; WO 00/42651; WO 01/75767 and U.S. Pat. No. 6,537,746. Other directed evolution procedures that can be used include, but are not limited to, staggered extension process (StEP), in vitro recombination (Zhao et al., 1998, Nat. Biotechnol. 16:258-261), mutagenic PCR (Caldwell et al., 1994, PCR Methods Appl. 3:S136-S140), and cassette mutagenesis (Black et al., 1996, Proc Natl Acad Sci USA 93:3525-3529).

The clones obtained following mutagenesis treatment are screened for engineered monoamine oxidases having a desired improved enzyme property. The measurement of enzymatic activity from the expression libraries can be performed using standard biochemistry techniques, such as, but not limited to using published methods or adaptations thereof for measuring monoamine oxidases, such as, but not limited to those methods disclosed by Zhou et al. (Zhou et al. “A One-Step Fluorometric Method for the Continuous Measurement of Monoamine Oxidase Activity,” 1997 Anal. Biochem. 253:169-74) and Szutowicz et al. (Szutowicz et al., “Colorimetric Assay for Monoamine Oxidase in Tissues Using Peroxidase and 2,2′-Azino (3-ethylbenzthaizoline-6-sulfonic Acid) as Chromogen,” 1984, Anal. Biochem. 138:86-94). Comparisons of enzymatic activities are made using a defined preparation of enzyme, a defined assay under a set condition, and one or more defined substrates, as further described in detail herein or using the methods of, e.g., Zhou and Szutowicz. Generally, when lysates are compared, the number of cells and the amount of protein assayed are determined, and identical expression systems and identical host cells are used to minimize variations in the amount of enzyme produced by the host cells and present in the lysates. In the case that the desired improved enzyme property is thermostability, enzymatic activity may be measured after subjecting the enzyme preparation to a defined temperature and measuring the amount of enzymatic activity remaining after heat treatment. Clones containing a polynucleotide encoding a monoamine oxidase are then isolated, sequenced to identify the nucleotide sequence changes (if any), and used to express the enzyme in a host cell.

In the case that the sequence of the engineered polypeptide is known, the polynucleotides encoding the enzyme can be prepared by standard solid-phase methods, according to known synthetic methods. In some embodiments, fragments of up to about 100 bases can be individually synthesized, and then joined (e.g., by enzymatic or chemical litigation methods, or polymerase mediated methods) to form any desired continuous sequence. For example, polynucleotides and oligonucleotides disclosed herein can be prepared by chemical synthesis using, e.g., the classical phosphoramidite method described by Beaucage et al., 1981, Tet Lett 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company; Midland, Tex., The Great American Gene Company, Ramona, Calif., ExpressGen Inc. Chicago, Ill., Operon Technologies Inc., Alameda, Calif., and many others.

Engineered monoamine oxidases expressed in a host cell can be recovered from the cells and/or the culture medium using any one or more of the well-known techniques for protein purification, including, but are not limited to, lysozyme treatment, sonication, filtration, salting-out, ultra-centrifugation, and chromatography. Suitable solutions for lysing and the high efficiency extraction of proteins from bacteria, such as E. coli, are commercially available under the trade name CclLytic B™ from Sigma-Aldrich of St. Louis Mo.

Chromatographic techniques for isolation of the monoamine oxidase include, but are not limited to, reverse phase chromatography high performance liquid chromatography, ion exchange chromatography; gel electrophoresis, and affinity chromatography. Conditions for purifying a particular enzyme will depend, in part, on factors such as net charge, hydrophobicity, hydrophilicity, molecular weight, molecular shape, etc., and will be apparent to those skilled in the art.

In some embodiments, affinity techniques may be used to isolate the improved monoamine oxidase. For affinity chromatography purification, any antibody which specifically binds the monoamine oxidase may be used. For the production of antibodies, various host animals, including but not limited to rabbits, mice, rats, etc., may be immunized by injection with a compound. The compound may be attached to a suitable carrier, such as BSA, by means of a side chain functional group or a linker attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette Guerin) and Corynebacterium parvum.

As is known by one skilled in the art, oxidase reactions catalyzed by monoamine oxidase typically require a cofactor. Oxidation reactions catalyzed by the monoamine oxidases described herein also typically require a cofactor, flavin-adenine nucleotide (FAD). As used herein, the term “cofactor” refers to a non-protein compound that operates in combination with a monoamine oxidase. Generally, the oxidized form of the cofactor, which may be non-covalently or covalently attached to the monoamine oxidase, is added to the reaction mixture. The oxidized FAD form can be regenerated from the reduced form FAD-H₂ by molecular oxygen. In another embodiment, the oxidized FAD form could be regenerated by NAD(P) to provide FAD and NAD(P)H. The NAD(P) could, in turn, be regenerated by reduction of a ketone to an alcohol using an NAD(P)H-dependent alcohol dehydrogenase/ketone reductase.

The oxidation reactions catalyzed by monoamine oxidase described herein are generally carried out in a solvent. Suitable solvents include water, organic solvents (e.g., ethyl acetate, butyl acetate, 1-octanol, heptane, octane, methyl t-butyl ether (MTBE), toluene, and the like), and ionic liquids (e.g., 1-ethyl 4-methylimidazolium tetrafluoroborate, 1-butyl-3-methylimidazolium tetrafluoroborate, 1-butyl-3-methylimidazolium hexafluorophosphate, and the like). In some embodiments, aqueous solvents, including water and aqueous co-solvent systems, are used.

Exemplary aqueous co-solvent systems comprise water and one or more organic solvents. In general, an organic solvent component of an aqueous co-solvent system is selected such that it does not completely inactivate the monoamine oxidase. Appropriate co-solvent systems can be readily identified by measuring the enzymatic activity of the specified engineered monoamine oxidase with a defined substrate of interest in the candidate solvent system, utilizing an enzymatic activity assay, such as those described herein.

The organic solvent component of an aqueous co-solvent system may be miscible with the aqueous component, providing a single liquid phase, or may be partly miscible or immiscible with the aqueous component, providing two liquid phases. Generally, when an aqueous co-solvent system is employed, it is selected to be biphasic, with water dispersed in an organic solvent, or vice-versa. Generally, when an aqueous co-solvent system is utilized, it is desirable to select an organic solvent that can be readily separated from the aqueous phase. In general, the ratio of water to organic solvent in the co-solvent system is typically in the range of from about 90:10 to about 10:90 (v/v) of organic solvent to water, and between 80:20 and 20:80 (v/v) of organic solvent to water. The co-solvent system may be pre-formed prior to addition to the reaction mixture, or it may be formed in situ in the reaction vessel.

The aqueous solvent (water or aqueous co-solvent system) may be pH-buffered or unbuffered. Generally, the oxidation can be carried out at a pH of about 10 or below, usually in the range of from about 5 to about 10. In some embodiments, the oxidation is carried out at a pH of about 9 or below, usually in the range of from about 5 to about 9. In some embodiments, the oxidation is carried out at a pH of about 8 or below, often in the range of from about 5 to about 8, and usually in the range of from about 6 to about 8. The oxidation may also be carried out at a pH of about 7.8 or below; or 7.5 or below. Alternatively; the oxidation may be carried out at neutral pH, i.e., about 7.

During the course of the oxidation reactions, the pH of the reaction mixture may change. Typical amines represented by structural Formula I are protonated at and about neutral pH, while the imine products represented by structural Formula II are typically not protonated at and about neutral pH. Accordingly, in typical embodiments wherein the reaction is conducted at or about neutral pH, the oxidation of the protonated amine to the un-protonated imine releases a proton into the aqueous solution. The pH of the reaction mixture may be maintained at a desired pH or within a desired pH range by the addition of a base during the course of the reaction. Alternatively, the pH may be controlled by using an aqueous solvent that comprises a buffer. Suitable buffers to maintain desired pH ranges are known in the art and include, for example, phosphate buffer, triethanolamine buffer, and the like. Combinations of buffering or base addition may also be used.

Suitable bases for neutralization of acids include organic bases, for example amines, alkoxides and the like, and inorganic bases, for example, hydroxide salts (e.g., NaOH), carbonate salts (e.g., NaHCO₃), bicarbonate salts (e.g., K₂CO₃), basic phosphate salts (e.g., K₂HPO₄, Na₃PO₄), and the like. A preferred base for neutralizing the protons released from oxidation of the amine to the imine over the course of the reaction is the amine substrate itself. The addition of a base concurrent with the course of the conversion may be carried out manually while monitoring the pH of the reaction mixture or, more conveniently; by using an automatic titrator as a pH stat. A combination of partial buffering capacity and base addition can also be used for process control. Typically, bases added to unbuffered or partially buffered reaction mixtures over the course of the oxidation are added in aqueous solutions.

During carrying out the stereoselective oxidation reactions described herein, the engineered monoamine oxidases may be added to the reaction mixture in the form of purified enzymes, whole cells transformed with gene(s) encoding the monoamine oxidase, and/or cell extracts and/or lysates of such cells. The whole cells transformed with gene(s) encoding the engineered monoamine oxidase or cell extracts and/or lysates thereof, may be employed in a variety of different forms, including solid (e.g., lyophilized, spray-dried, and the like) or semisolid (e.g., a crude paste).

The cell extracts or cell lysates may be partially purified by precipitation (ammonium sulfate, polyethyleneimine, heat treatment or the like), followed by a desalting procedure (e.g., ultrafiltration, dialysis, and the like) prior to lyophilization. Any of the cell preparations may be stabilized by crosslinking using known crosslinking agents, such as, for example, glutaraldehyde, or by immobilization to a solid phase (e.g., Eupergit C, and the like).

Solid reactants (e.g., enzyme, salts, etc.) may be provided to the reaction in a variety of different forms, including powder (e.g., lyophilized, spray-dried, and the like), solution, emulsion, suspension, and the like. The reactants can be readily lyophilized or spray-dried using methods and equipment that are known to those of ordinary skill in the art. For example, the protein solution can be frozen at −80° C. in small portions, then added to a prefrozen lyophilization chamber, followed by the application of a vacuum. After the removal of water from the samples, the temperature is typically raised to 4° C. for two hours before release of the vacuum and retrieval of the lyophilized samples.

The amount of reactants used in the oxidation reaction will generally vary depending on the amount of products desired, and concomitantly the amount of the monoamine oxidase substrate employed. Generally; substrates can be employed at a concentration of about 5 g/L to 50 g/L when monoamine oxidase is used at about 50 mg/L to about 5 g/L. Those of ordinary skill in the art will readily understand how to vary these amounts to tailor them to the desired level of productivity and scale of production. Appropriate amount of optional agents, such as catalase, antifoamer, and sodium bisulfite or sodium metabisulfite may be readily determined by routine experimentation.

The order of addition of reactants is not strict. The reactants may be added together at the same time to a solvent (e.g., monophasic solvent, biphasic aqueous co-solvent system, and the like), or alternatively, some of the reactants may be added separately with some being added together, at different time points. In certain embodiments, one or more of the components of the reaction may be added (“fed”) continuously to the reaction at levels that minimize or obviate substrate and/or product inhibition of the monoamine oxidase. In certain embodiments, the monoamine oxidase can be added at intervals over the course of the reaction, for example added at about every 1 hour, about every 2 hours, about every 3 hours, or about every 4 hours.

Suitable conditions for carrying out the monoamine oxidase-catalyzed oxidation reactions described herein include a wide variety of conditions which can be readily optimized by routine experimentation that includes, but is not limited to, contacting the engineered monoamine oxidase and substrate at an experimental pH and temperature, and detecting products, for example, using the methods described in the Examples provided herein.

The monoamine oxidase-catalyzed oxidation is typically carried out at a temperature in the range of from about 5° C. to about 75° C. For some embodiments, the reaction is carried out at a temperature in the range of from about 20° C. to about 55° C. In other embodiments, the reaction is carried out at a temperature in the range of from about 20° C. to about 45° C., about 30° C. to about 45° C., or about 40° C. to about 45° C. The reaction may also be carried out under ambient conditions (about 21° C.).

The oxidation reaction is generally allowed to proceed until essentially complete or nearly complete oxidation of substrate is obtained. Oxidation of substrate to product can be monitored using known methods by detecting substrate and/or product. Suitable methods include gas chromatography, HPLC, and the like. Conversion yields are generally greater than about 50%, may also be greater than about 60%, may also be greater than about 70%, may also be greater than about 80%, may also be greater than 90%, and are generally greater than about 97%.

All publications and patent applications mentioned herein are incorporated herein by reference to the same extent as if each individual publication or patent application was specifically and individually indicated to be incorporated by reference.

### BRIEF DESCRIPTION OF DRAWINGS

FIG. 1 represents the standard curve of H₂O₂ concentration detected by a fluorescence method using Ampliflu™ Red.

FIG. 2 represents enzymatic activity assay results of MAON mutants.

| No. | Name |
|---|---|
| SEQ ID NO: 1 | Monoamine oxidase MAON |
| SEQ ID NO: 2 | MAON mutant F63L |
| SEQ ID NO: 3 | MAON mutant T65V |
| SEQ ID NO: 4 | MAON mutant S100P |
| SEQ ID NO: 5 | MAON mutant T141S |
| SEQ ID NO: 6 | MAON mutant S234C |
| SEQ ID NO: 7 | MAON mutant F63L/T65V |
| SEQ ID NO: 8 | MAON mutant S234C/S100P |
| SEQ ID NO: 9 | MAON mutant S234C/T141S |
| SEQ ID NO: 10 | MAON mutant F63L/T65V/S100P |
| SEQ ID NO: 11 | MAON mutant F63L/T65V/T141S |
| SEQ ID NO: 12 | MAON mutant F63L/T65V/S234C |
| SEQ ID NO: 13 | MAON mutant S234C/T141S/S100P |
| SEQ ID NO: 14 | MAON mutant F63L/T65V/S100P/T141S |
| SEQ ID NO: 15 | MAON mutant F63L/T65V/S100P/S234C |
| SEQ ID NO: 16 | MAON mutant F63L/T65V/T141S/S234C |
| SEQ ID NO: 17 | MAON mutant F63L/T65V/S100P/T141S/S234C |

### DETAILED EMBODIMENTS OF THE INVENTION

Various features and embodiments of the present disclosure are illustrated in the following representative examples, which are intended to be illustrative, and not limiting.

### Example 1 Construction of Vectors of Monoamine Oxidase Mutants

The monoamine oxidase MAON protein expression gene originated from Aspergillus niger and the MAON mutant gene (SEQ ID NO: 1-17) modified and designed based on stability and catalytic activity were ligated to the E. coli expression vector pET15b and inserted at the NdeI+BamHI sites, and the N-ter 6×His tag was preserved. After sequenced as correct, the recombinant vector was transformed into BL21 (DE3) for protein expression.

### Example 2 Expression and Purification of Monoamine Oxidases: Production by Shake Flask

The constructed expression vectors were transformed into E. coli BL21 (DE3) and induced by IPTG for expression. The bacteria were harvested, lysed and purified by a Ni-NTA column. The specific method is as follows: the MAON enzyme recombinant expression vectors were transformed into BL21 (DE3) strain. A single clone was picked into 10 mL of LB medium with ampicillin sodium resistance (100 mg/L) and cultured overnight at 37° C. and 200 rpm. The culture was transferred to a 2-L flask containing 1 L of LB medium, cultured at 37° C. and 200 rpm until the OD₆₀₀ reached 0.6-0.8, and then cooled to 25° C., induced by 0.5 mM IPTG for expression overnight, and centrifuged at 5000×g to collect the cells. The collected cells were resuspended in buffer A: 50 mM Tris pH 8.0, 500 mM NaCl and 20 mM imidazole, added with PMSF with a final concentration of 1 mM and 250 μL of Cocktail inhibitor, and mixed evenly. After being crushed by a high-pressure homogenizer, the culture was centrifuged at 43000×g and 4° C. for 30 min, and the supernatant was collected and passed through a Ni column. Purification via a Ni-NTA column was carried out where the lysate supernatant was combined with the resin for 20 min, then washed with buffer A containing 50 mM imidazole to remove impurities, and finally eluted with an elution buffer containing 400 mM imidazole. The purification effect of protein was detected by SDS-PAGE. Dialysis was performed by changing the buffer to 50 mM Tris pH 7.5, 500 mM NaCl, and 1 mM DTT. The final sample was detected for the purification effect of the protein by SDS-PAGE, and after ultrafiltration, frozen and stored at −80° C. for future use.

### Example 3 Expression and Purification of Monoamine Oxidases: Fermentation Production

Seed activation: the MAON enzyme recombinant expression vectors were transformed into BL21 (DE3) strain. A single clone was picked into 10 mL of LB medium with ampicillin sodium resistance (100 mg/L), and cultured overnight at 37° C. and 200 rpm. The culture was transferred into a 1-L flask containing 500 mL of LB medium and cultured at 37° C. and 200 rpm until the OD₆₀₀ reached 0.8-1.0. Fermentation culture: A 10-L fermenter containing 6 L of TB medium was preheated to 37° C., added with ampicillin sodium with a final concentration of 100 mg/L, and after inoculation, aerated and stirred to maintain 30% dissolved oxygen. When OD₆₀₀ reached 10, feeding was started, wherein feed 1 was an aqueous solution containing 60 g/L tryptone, 120 g/L yeast extract and 4% glycerin, and feed 2 was 50% glycerin. Ammonia water and phosphoric acid were employed to adjust the pH such that the pH was stabilized at 7.0. When OD₆₀₀ reached 20, the fermentation broth was cooled to 25° C. added with isopropyl-β-D-thiogalactoside (IPTG) with a final concentration of 1 mM to induce the expression of monoamine oxidases, and the culture was grown for another 20 hours until being harvested. The culture was centrifuged at 8000×g to collect cells. The harvested cells were directly used in the subsequent purification process or stored at −80° C. until used as such. Crude enzyme purification: the collected cells were resuspended using 100 mM Tris pH 8.0 and 150 mM NaCl to 200 g of wet cells/L and mixed well. After being crushed by a high-pressure homogenizer under 800 bar, the culture was centrifuged at 18400×g and 4° C. for 20 min. The supernatant was taken and added with ammonium sulfate powder with a final concentration of 36% saturation (200 g/L) and centrifuged to collect protein precipitate. The precipitate was stored at 4° C. for later use after being lyophilized.

### Example 4 Enzymatic Activity Assay of Monoamine Oxidases

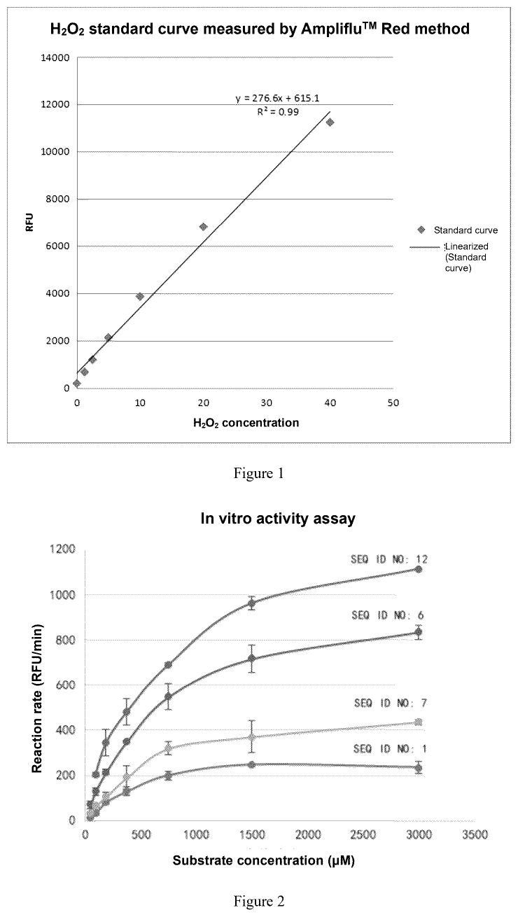

Since monoamine oxidases catalyze the production of H₂O₂, the enzymatic activity parameters can be measured indirectly by measuring the amount of hydrogen peroxide produced. To draw a standard curve: 5 mL of 100 mM K₂HPO₄·HCl buffer at pH 7.4 was taken and added with Ampliflu™ Red dye with a final concentration of 100 μM and 1 U/mL horseradish peroxidase to prepare a working solution, which was used for separately preparing reaction solutions containing H₂O₂ at concentrations of 0, 1.25, 2.5, 5, 10, 20, and 40. Fluorescence values were measured by a microplate reader (λₑₓ=535 nm/λₑₘ=590 nm) and the standard curve was drawn as shown in FIG. 1. MAON enzymatic activity parameters were measured using 100 mM K₂HPO+·HCl buffer at pH 7.4. The method described in Example 2 was adopted to purify the resulting MAON enzyme which was determined for protein concentrations by BCA method and diluted into a 50 nM working solution. The buffer was used to prepare the substrate to reaction solutions with final concentrations of 0, 47, 94, 188, 375, 750, 1500, and 3000 μM. 95 μL of the reaction solution was taken and added with 5 μL of the enzyme working solution. The final enzyme working concentration was 2.5 nM. The changes in fluorescence values were measured by a microplate reader (λₑₓ=535 nm/λₑₘ=590 nm) and some test results are as shown in FIG. 2. As shown in the FIG. 2, the enzymatic activity of the SEQ ID NO: 7 mutant is 2 times that of the control SEQ ID NO: 1, the enzymatic activity of the SEQ ID NO: 6 mutant is 4 times that of the control SEQ ID NO: 1, the enzymatic activity of the SEQ ID NO: 12 mutant is 5 times that of the control SEQ ID NO: 1.

### Example 5 Activity Assay of Monoamine Oxidase-Catalyzed Oxidation of 6,6-dimethyl-3-azabicyclo[3.1.0]hexane to Produce (1R,5S)-6,6-dimethyl-3-azabicyclo[3.1.0]hex-2-ene

40 mL of a buffer of the monoamine oxidase of SEQ ID NO: 1-17 (2 mg/mL, stored in potassium phosphate-hydrochloric acid buffer, pH 7.4, yellow liquid) which was expressed and purified in Example 2 was added into a three-necked flask, added with 70 mg of catalase and 2 mg of antifoamer 204, and stirred at 25° C. in an oxygen environment. In addition, 280 mg of 6,6-dimethyl-3-azabicyclo[3.1.0]hexane was weighed and dissolved with 5 mL of potassium phosphate-hydrochloric acid buffer at pH 7.4, and then the mixture was added dropwise to the aforementioned reaction system through a syringe pump within 5 hours. During the reaction, 3 M sodium hydroxide solution was used to adjust the pH to remain at 7.4. After 18 hours of reaction, in-process control was performed by LC-MS and the conversion rate was >95%. Upon the completion of the reaction, methyl tert-butyl ether was added for extraction, which was then spin-dried to obtain the product, and the ee value of the liquid phase was detected as >99%.

¹H NMR (300 MHz, Chloroform-d) δ 7.39-7.35 (m, 1H), 3.92-3.81 (m, 1H), 3.62-3.52 (m, 1H), 2.18-1.99 (m, 1H), 1.72-1.63 (m, 1H), 1.10 (s, 3H), 0.76 (s, 3H).

### Example 6 Monoamine oxidase-catalyzed desymmetrization of 6,6-dimethyl-3-azabicyclo[3.1.0]hexane in the Presence of bisulfite

40 mL of potassium phosphate-hydrochloric acid buffer at pH 7.4 was added into a three-necked flask, added with 500 mg of the monoamine oxidase of SEQ ID NO: 1-17 expressed and purified in Example 2, then added with 175 mg of catalase and 2.5 mg of antifoamer 204, and stirred at 25° C. in an oxygen environment. In addition, 1.25 g of sodium bisulfite was weighed and dissolved with 8 mL of water, and then added with 1 g of 6,6-dimethyl-3-azabicyclo[3.1.0]hexane. The substrate was added dropwise to the bio-enzyme reaction system through a syringe pump within 5 hours. During the reaction, 3 M sodium hydroxide solution was used to adjust pH to remain at 7.4. After 24 hours, in-process control was performed, and the LC-MS analysis showed that the conversion of raw material was completed. The system was a mixture of (1R,5S)-6,6-dimethyl-3-azabicyclo[3.1.0]hex-2-ene and (1R,2S,5S)-6,6-dimethyl-3-azabicyclo[3.1.0]hexane-2-sodium sulfonate. Wherein, samples were taken and detected when the reaction progressed to 1 h, 3 h, 6 h, 9 h, and 11 h, respectively, showing that the remaining amount of substrate in the reaction systems of SEQ ID NO: 6, SEQ ID NO: 7, and SEQ ID NO: 12 samples was less than that of SEQ ID NO: 1 sample, which indicated higher activity.

### Example 7 Preparation of (1R,2S,5S)-6,6-dimethyl-3-azabicyclo[3.1.0]hexane-2-carbonitrile

40 mL of potassium phosphate-hydrochloric acid buffer at pH 7.4 was added into a three-necked flask, added with the monoamine oxidase of SEQ ID NO: 1-17 expressed and purified in Example 2, then added with 175 mg of catalase and 2.5 mg of antifoamer 204, and stirred at 25° C. in an oxygen environment. In addition, 1.25 g of sodium bisulfite was weighed and dissolved with 8 mL of water, and then added with 1 g of 6,6-dimethyl-3-azabicyclo[3.1.0]hexane. The substrate was added dropwise to the bio-enzyme reaction through a syringe pump within 5 hours. During the reaction, 3 M sodium hydroxide solution was used to adjust pH to remain at 7.4. After 24 hours, in-process control was performed, and the LC-MS analysis showed that the conversion of raw material was completed. The system was a mixture of (1R,5S)-6,6-dimethyl-3-azabicyclo[3.1.0]hex-2-ene and (1R,2S,5S)-6,6-dimethyl-3-azabicyclo[3.1.0]hexane-2-sodium sulfonate. The reaction system was cooled to 10° C., then added with 40 mL of MTBE, and dropwise added with 1 g of TMSCN within 30 min, and the LC-MS analysis indicated product formation after reaction for 30 min. Wherein, after the reaction solution in the reaction system of SEQ ID NO: 6 sample was filtered through diatomaceous earth, the MTBE phase and the aqueous phase were separated. The aqueous phase was extracted three times with MTBE, and then the MTBE phases were combined, dried over sodium sulfate and spin-dried to obtain 950 mg of the product, with a yield of 77%.

While the present invention is satisfied by embodiments in many different forms, as described in detail in conjunction with preferred embodiments of the present invention, it is understood that the present disclosure is to be considered as exemplary of the principles of the present invention and is not intended to limit the present invention to the specific embodiments illustrated and described herein. Numerous variations may be made by persons skilled in the art without departing from the spirit of the present invention. The scope of the present invention will be determined by the appended claims and their equivalents. The abstract and the title are not to be construed as limiting the scope of the present invention, as their purpose is to enable the appropriate authorities, as well as the general public, to quickly determine the general nature of the present invention.

1. A monoamine oxidase, comprising an amino acid sequence having a mutation compared with the amino acid sequence of SEQ ID NO: 1, wherein the mutation is selected from the group consisting of: a mutation of the amino acid at position 63 from phenylalanine into leucine, a mutation of the amino acid at position 65 from threonine into valine, a mutation of the amino acid at position 100 from serine into proline, a mutation of the amino acid at position 141 from threonine into serine, a mutation of the amino acid at position 234 from serine into cysteine, and a combination thereof, in an amino acid sequence corresponding to the amino acid sequence of SEQ ID NO: 1.

2. The monoamine oxidase according to claim 1, wherein the monoamine oxidase comprises an amino acid sequence having a mutation compared with the amino acid sequence of SEQ ID NO: 1, wherein the mutation is selected from the group consisting of:
in an amino acid sequence corresponding to the amino acid sequence of SEQ ID NO: 1, a mutation of the amino acid at position 63 from phenylalanine into leucine and a mutation of the amino acid at position 65 from threonine into valine;
in an amino acid sequence corresponding to the amino acid sequence of SEQ ID NO: 1, a mutation of the amino acid at position 100 from serine into proline and a mutation of the amino acid at position 234 from serine into cysteine;
in an amino acid sequence corresponding to the amino acid sequence of SEQ ID NO: 1, a mutation of the amino acid at position 141 from threonine into serine and a mutation of the amino acid at position 234 from serine into cysteine;
in an amino acid sequence corresponding to the amino acid sequence of SEQ ID NO: 1, a mutation of the amino acid at position 63 from phenylalanine into leucine, a mutation of the amino acid at position 65 from threonine into valine, and a mutation of the amino acid at position 100 from serine into proline;
in an amino acid sequence corresponding to the amino acid sequence of SEQ ID NO: 1, a mutation of the amino acid at position 63 from phenylalanine into leucine, a mutation of the amino acid at position 65 from threonine into valine, and a mutation of the amino acid at position 141 from threonine into serine;
in an amino acid sequence corresponding to the amino acid sequence of SEQ ID NO: 1, a mutation of the amino acid at position 63 from phenylalanine into leucine, a mutation of the amino acid at position 65 from threonine into valine, and a mutation of the amino acid at position 234 from serine into cysteine;
in an amino acid sequence corresponding to the amino acid sequence of SEQ ID NO: 1, a mutation of the amino acid at position 100 from serine into proline, a mutation of the amino acid at position 141 from threonine into serine, and a mutation of the amino acid at position 234 from serine into cysteine;
in an amino acid sequence corresponding to the amino acid sequence of SEQ ID NO: 1, a mutation of the amino acid at position 63 from phenylalanine into leucine, a mutation of the amino acid at position 65 from threonine into valine, a mutation of the amino acid at position 100 from serine into proline, and a mutation of the amino acid at position 141 from threonine into serine;
in an amino acid sequence corresponding to the amino acid sequence of SEQ ID NO: 1, a mutation of the amino acid at position 63 from phenylalanine into leucine, a mutation of the amino acid at position 65 from threonine into valine, a mutation of the amino acid at position 100 from serine into proline, and a mutation of the amino acid at position 234 from serine into cysteine;
in an amino acid sequence corresponding to the amino acid sequence of SEQ ID NO: 1, a mutation of the amino acid at position 63 from phenylalanine into leucine, a mutation of the amino acid at position 65 from threonine into valine, a mutation of the amino acid at position 141 from threonine into serine, and a mutation of the amino acid at position 234 from serine into cysteine; and
in an amino acid sequence corresponding to the amino acid sequence of SEQ ID NO: 1, a mutation of the amino acid at position 63 from phenylalanine into leucine, a mutation of the amino acid at position 65 from threonine into valine, a mutation of the amino acid at position 100 from serine into proline, a mutation of the amino acid at position 141 from threonine into serine, and a mutation of the amino acid at position 234 from serine into cysteine.

3. The monoamine oxidase according to claim 1, wherein the amino acid sequence of the monoamine oxidase further has at least 80%, at least 85%, at least 90%, at least 95%, at least 96%, at least 97%, at least 98%, or at least 99% sequence identity to the amino acid sequence of SEQ ID NO: 1.

4. The monoamine oxidase according to claim 1, wherein the monoamine oxidase comprises an amino acid sequence that has at least 92%, at least 95%, at least 96%, at least 97%, at least 98%, at least 99% or 100% sequence identity to any amino acid sequence selected from the group consisting of SEQ ID NOs: 2-17.

5. The monoamine oxidase according to claim 4, wherein the amino acid sequence of the monoamine oxidase is as set forth in any one of SEQ ID NOs: 2-17.

6. A polynucleotide encoding the monoamine oxidase according to claim 1.

7. A method for producing a compound as represented by Formula II or a salt/hydrate thereof, comprising contacting a compound as represented by Formula I with oxygen in the presence of the monoamine oxidase according to claim 1 and a cofactor;

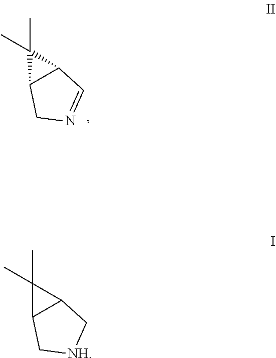

8. A method for producing an aminosulfonate compound as represented by Formula III or a salt/hydrate thereof, comprising contacting a compound as represented by Formula I with oxygen in the presence of the monoamine oxidase according to claim 1, a cofactor and bisulfite;

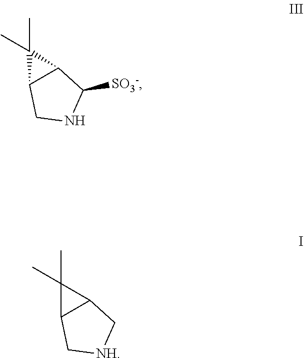

9. A method for producing an aminonitrile compound as represented by Formula IV or a salt/hydrate thereof, comprising contacting a compound as represented by Formula I with oxygen in the presence of the monoamine oxidase according to claim 1, a cofactor and bisulfite to obtain an aminosulfonate compound and contacting the aminosulfonate compound with cyanide;

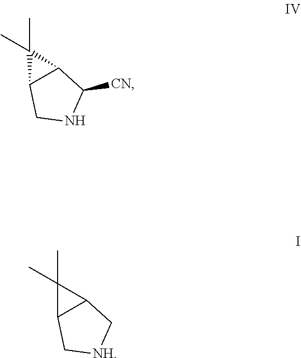

10. The method according to claim 7,
wherein the cofactor is non-covalently associated with a monoamine oxidase.

11. (canceled)

12. (canceled)

13. (canceled)

14. A host cell comprising the polynucleotide according to claim 6.

15. The method according to claim 10, wherein the cofactor is selected from the group consisting of FAD, FMN, NAD and NADP.

16. The method according to claim 10, wherein the method further comprises a component catalyzing the disproportionation of hydrogen peroxide to molecular oxygen and water; more preferably, the component is selected from the group consisting of Pd, Fe and catalase.
